(19) United States
(12) Patent Application Publication
(10) Pub. No.: US 2013/0059309 A1
(43) Pub. Date: Mar. 7, 2013

(54) Dopaminergic Neurons Derived From Induced Pluripotent Stem Cells and Method of Making Same

(71) Applicant: Jian Feng, Getzville, NY (US)

(72) Inventor: Jian Feng, Getzville, NY (US)

(21) Appl. No.: 13/580,025

(22) PCT Filed: Jan. 28, 2011
(86) PCT No.: PCT/US11/22897

§ 371 Date: Nov. 20, 2012

Related U.S. Application Data
(60) Provisional application No. 61/306,805, filed on Feb. 22, 2010.

(51) Int. Cl. C12N 5/0793; G01N 33/566; C12Q 1/04; C12N 15/87; C12Q 1/68

(52) U.S. Cl. 435/6.12; 435/325; 435/455; 435/377; 435/6.13; 435/34; 435/7.21

(57) ABSTRACT

Provided are compositions and methods that relate to cultured neurons. The cultured neurons can either have or not have genetic mutations that are characteristic of Parkinson's disease (PD). The cultured neurons are generated from induced pluripotent stem cells made from human fibroblasts that are obtained from individuals with and without PD. Cultured neurons without genetic mutations characteristic of PD are dopaminergic neurons that exhibit specific dopamine uptake are provided. Also provided is a method for identifying a test agent as a potential candidate for reducing the severity of PD. The method involves obtaining cells of neural lineage derived from cells obtained from an individual who has PD and measuring the effects of the test agents on dopaminer-characteristics, including specific dopamine uptake, monoamine oxidase (MAO) transcription levels, protein and/or activity levels of estrogen-related receptors, and combinations thereof. An increase in specific dopamine uptake, inhibition of MAO transcription or decrease in the level and/or activity of estrogen-related receptors caused by the agent can be used to identify the agent as a potential candidate for reducing the severity of PD.

### CROSS-REFERENCE TO RELATED APPLICATIONS

This application claims priority to U.S. patent application No. 61/306,805, filed Feb. 22, 2010, the disclosure of which is incorporated herein by reference.

### STATEMENT REGARDING FEDERALLY SPONSORED RESEARCH OR DEVELOPMENT

This invention was made with government support under RC2NS070276 awarded by National Institutes of Health. The government has certain rights in the invention.

### FIELD OF THE INVENTION

The present invention relates generally to induced pluripotent stem cells derived from fibroblasts obtained from normal individuals and from familial Parkinson's patients, and method for differentiating the iPSCs into dopaminergic neurons.

### BACKGROUND OF THE INVENTION

iPSC are a type of pluripotent stem cell artificially derived from a non-pluripotent cell, typically an adult somatic cell, by inducing a “forced” expression of certain genes. iPS cells are thus derived from adult somatic cells (e.g. skin fibroblasts) (a) via transgenes introduced by viral vectors or plasmids or (b) by small molecules or certain proteins that can reprogram the cells to become pluripotent.

iPS cells can potentially provide cell types that would avoid the need to use controversial, embryonic stem cells for uses such as the study of disease and screening of drug candidates. However, it has not been possible to derive certain types of cells having desirable phenotypes from existing methods for generating and differentiating iPSCs. For example, to the best of our knowledge no one has previously been able to develop dopaminergic neurons that exhibit physiologically relevant dopamine uptake and it has accordingly not been possible to use cultured neurons for screening for compounds that modulate dopamine uptake by such neurons. This has hampered drug development for neurological disorders for diseases like Parkinson's disease (PD), which has two main forms, sporadic/idiopathic and familial/genetic. Sporadic/idiopathic PD is the most common form, but the cause is unclear. Some research indicates that it could be environmental related, perhaps caused by exposure to toxins, and idiopathic PD is hard to study for these reasons. Irrespective of what the potential causes of idiopathic PD may be, it is expected that because of its diverse etiology no single therapeutic approach is near. In contrast, a major cause of Familial/Genetic PD is fairly well understood as being caused by parkin gene mutations. Familial PD is less common than the idiopathic form, with 5 to 15% of all cases of PD falling in this category. Pathophysiology of PD involves loss of dopaminergic neurons in substantia nigra, with symptoms including: bradykinesia, rigidity, tremors, instability and lack of speech control. Symptoms get progressively worse with time. In the United States, at least 500,000 people are believed to suffer from Parkinson's disease, and about 50,000 new cases are reported annually. These figures are expected to increase as the average age of the population increases. If 5-15% of those patients have familial/genetic PD, that means that 25,000-75,000 people suffer from familial PD in the U.S. Thus, there is an ongoing need for cell populations comprising dopaminergic neurons that can be used for testing potential therapeutic agents for conditions including PD.

### SUMMARY OF THE INVENTION

The present invention provides compositions and methods that relate to cultured dopaminergic neurons derived from induced pluripotent stem cells (iPSCs) with or without genetic mutations characteristic of familial Parkinson's disease (PD). Cultured dopaminergic neurons without genetic mutations characteristic of familial PD unexpectedly exhibit specific dopamine uptake.

In one embodiment, the invention provides iPSCs for use in generating cultured cells of neuronal lineage. The iPSCs can be made using fibroblasts obtained from individuals who do not have familial PD. Accordingly, the present invention also provides a method for forcing the differentiation of the iPSCs toward a neuronal cell lineage to obtain dopaminergic neuronal cells. The methods of obtaining the iPSCs and the method of differentiation of the iPSCs into cells of neuronal lineage can be applied to fibroblasts obtained from normal individuals or from individuals with PD.

The method for generating iPSCs from cells obtained from individuals with or without PD generally comprises obtaining fibroblasts from the individual and introducing Oct4, Sox2, Klf4, c-Myc and Nanog genes into the fibroblasts. The invention also comprises introducing a tetracycline-controlled transactivator (M2rtTAA) into the fibroblasts. Each of the Oct4, Sox2, Klf4, c-Myc, Nanog and M2rtTAA can be flanked by Lox sites and thus are removable from the cells if desired using Cre-Lox recombination techniques.

The method for differentiating the iPSCs into cells of neuronal lineage generally comprises generating the iPSCs from fibroblasts and plating cells on a surface coated with materials such that cells of neuronal lineage are generated. If iPSCs derived from an individual who does not have familial PD are used, cells of neuronal lineage can become dopaminergic neurons capable of specific dopamine uptake.

The invention also provides a method for identifying a test agent as a potential candidate for reducing the severity of PD. The method comprises obtaining cells of neural lineage derived from cells obtained from an individual who has PD and testing the effect of the test agent on various dopaminergic characteristics, include specific dopamine uptake, MAO transcription levels, protein and/or activity levels of estrogen-related receptors, and combinations thereof. Based on changes in dopaminergic characteristics in the samples, the agent is identified as a potential candidate for reducing the severity of PD. In particular, an increase in specific dopamine uptake, inhibition of MAO transcription or decrease in the level and/or activity of estrogen-related receptors caused by the agent can be used to identify the agent as a potential candidate for reducing the severity of PD.

### BRIEF DESCRIPTION OF THE DRAWINGS

FIG. 1 provides a graphical representation of specific dopamine (DA) uptake normalized against the amount of endogenous dopamine in the neuronal culture. It is apparent from the data that selective dopamine uptake is greatly diminished in iPSC-derived neurons from PD patients with parkin mutations, compared to that in controls. * denotes p<0.05, vs. any of the two control bars.

FIG. 2 provides a graphical summary of results obtained from analyzing MAO transcription. It is apparent from the data that monoamine oxidase (MAO) transcription is greatly increased in iPSC-derived neurons from PD patients with parkin mutations, compared to that in controls. **, p<0.01, vs. controls.

### DETAILED DESCRIPTION OF THE INVENTION

The present invention provides a method for obtaining iPSCs from fibroblasts, which iPSCs are capable of differentiating in vitro into dopaminergic cells exhibiting specific dopamine uptake. The present invention also provides a method for forcing the differentiation of the iPSCs toward neuronal cell lineage to obtain dopaminergic neuronal cells. The methods of obtaining the iPSCs and the method of differentiation of the iPSCs into cells of neuronal lineage can be applied to fibroblasts obtained from normal individuals or from individuals with PD. The term “normal individuals” or “control individuals” as used herein means individuals who do not have PD.

The present invention also provides cell populations comprising iPSCs derived from fibroblasts from normal individuals and from individuals with PD. Additionally, cells of neuronal lineage derived from the iPSCs are also provided. The cells of neuronal lineage obtained from iPSCs derived from control individuals display characteristics of dopaminergic neurons. In one embodiment are also provided cell populations comprising cells of neuronal lineage derived from iPSCs that are generated using cells obtained from an individual who has familial PD. These cells of neuronal lineage derived from iPSCs from PD patients with parkin mutations are referred to herein as “parkin neurons.” Parkin neurons comprise the same parkin mutation that is present in the individual from which the cells used to make the iPSCs are obtained. Thus, in various embodiments, the invention provides populations of cells that comprise either control neurons or parkin neurons. These cell populations can comprise, consist of or consist essentially of either control neurons or parkin neurons.

In one embodiment, the invention provides a population of cells comprising control neurons, wherein at least 1%, 2%, 3%, 4%, 5%, 6%, 7%, 8%, 9% or 10%, or more of the control neurons are dopaminergic neurons. The dopaminergic neurons exhibit specific dopamine uptake.

The invention is useful for screening test agents for potential use as therapeutic agents for treating diseases which are positively correlated with the presence of defective dopaminergic neurons in the brain of an individual. In this regard, we unexpectedly produced iPSC-derived dopaminergic control neurons that exhibit specific dopamine uptake. It is believed that the invention provides the first in vitro demonstration of neuronal cells (our “control neurons”) that have a phenotype characterized by specific dopamine uptake. These cells are of particular value because they can serve as a reference against which studies with defective cultured neurons (such as parkin neurons) can be compared. The control neurons can also be used directly to analyze the effect of test agents on dopamine uptake in cultured neurons that have functional dopamine uptake.

The parkin neurons provided by the invention are also useful for screening test agents or other factors that can modulate parkin neuron function. For instance, the effect of dopamine uptake by test agents on the parkin neurons can be compared to dopamine uptake by parkin neurons that have not been exposed to the test agent. It is considered that if the test agent increases specific uptake of dopamine by the parkin neurons, the test agent is a candidate for treating PD. Additionally, the control neurons can be used to set a reference level for a desired amount of specific dopamine uptake that test agents can induce in the parkin neurons. The amounts of specific and non-specific dopamine uptake can be determined using any of a variety of techniques that are used to directly or indirectly measure specific and non-specific dopamine uptake by cultured neurons.

In one embodiment, the invention provides a method for determining whether a test agent is a candidate for therapy of familial PD by determining whether the test agent can inhibit transcription of a monoamine oxidase in a population of cells comprising parkin neurons. This method is described more fully below.

Methods for obtaining iPSCs which have the ability to differentiate into neural lineage cells, and methods for differentiating the iPSCs to neuronal cells, particularly dopaminergic neuronal cells are as follows.

Generation of iPSCs.

The iPSCs are derived from adult somatic cells from individuals. In one embodiment, the adult somatic cells are fibroblasts. The fibroblasts can be obtained from human individuals of any age by techniques well known in the art. Generally, skin fibroblasts are easily obtained and methods for obtaining skin fibroblasts are well known in the art. In addition, embryonic stem cells can also be used. For example, fibroblasts can be obtained from normal individuals or from individuals diagnosed with Parkinson's disease (idiopathic or familial).

A part of our approach for making iPSCs was to modify the genetic engineering steps described in a previous publication (Soldner F, et al. Cell. 2009 Mar. 6;136(5):964-77). Importantly, the iPSCs described in Soldner et al. were generated from fibroblasts obtained from individuals with idiopathic PD and were not reported to form dopaminergic neurons exhibiting specific dopamine uptake. Those neurons, therefore, would not be useful in screening for test agents that could enhance specific dopamine uptake for treating PD.

To make iPSCs from fibroblasts, pluripotency genes are introduced by techniques well known in the art. In one embodiment, the genes are introduced via use of lentivirus. Such genes include, but are not limited to, Oct4, Sox2, Klf4 and c-Myc. In the present method, all of these genes and an additional gene, Nanog, were introduced in the fibroblasts. The method of Soldner et al. did not include insertion of a gene encoding Nanog. Preferably, the undifferentiated iPSCs have the capacity to divide and proliferate indefinitely in culture.

The nucleotide sequences encoding the reprogramming factors we used are known in the art and are described in GenBank. Each of the reprogramming factors for which a GenBank entry is provided is incorporated herein by reference as that entry existed on the priority date of this application. Those skilled in the art will know how to use GenBank's history of revisions if necessary to ascertain the nucleotide and amino acid sequences of the reprogramming factors that can be used in the invention. In particular, hSOX2 is described in GenBank entry NM_{—}003106. hKlf4 is described in GenBank entry NM_{—}004235. hc-Myc is described in GenBank entry NM_{—}002467. hNanog is described in GenBank entry NM_{—}024865. We also inserted the tetracycline-controlled transactivator referred to as M2rtTA. The M2rtTA sequence is known in the art and polynucleotides such as plasmids which comprise the M2rtTA sequence are commercially available from, for example, Addgene of Cambridge, Mass., USA (Addgene plasmid 20342).

Preferably, the iPSCs can be maintained in culture indefinitely in standard iPS cell culture media including Dulbecco's Modified Eagles Medium (DMEM)/F12 with 20% knockout serum replacement, 0.1 mM 2-mercaptoethanol, 0.1 mM MEM nonessential amino acids, 1 mM L-glutamine and 4 ng/ml FGF2. In one embodiment, the cells can be maintained at least for one year. In addition, the cells can be frozen for later use.

### Preparation of Dopaminergic Neurons

The iPSCs can be induced to differentiate in culture. As used herein, the term “differentiation” refers to a process whereby undifferentiated iPSCs acquire a more specialized fate, such as the fate of dopaminergic neurons. The term “dopaminergic neurons” as used herein means neuronal cells that exhibit certain characteristics of dopamine neurons, including at least specific uptake of dopamine. The dopaminergic neurons can exhibit specific uptake of dopamine that is from 50%-95%, inclusive, and including all integers and ranges there between, over endogenous levels of dopamine. In one embodiment, the dopaminergic neurons of the invention exhibit specific uptake of dopamine that is at least at least 70% over endogenous levels of dopamine. In one embodiment, the dopaminergic neurons exhibit specific uptake of dopamine that is at least at least 70% over endogenous levels of dopamine in approximately 5-15 minutes. Accordingly, the dopaminergic neurons of the invention can exhibit specific uptake of dopamine that is at least 70% over endogenous levels of dopamine within 5, 6, 7, 8, 9, 10, 11, 12, 13, 14, or 15 minutes. Endogenous dopamine means the amount of dopamine in the cultured neurons without addition of dopamine to the culture.

To induce differentiation of iPSCs into dopaminergic neurons, the iPSCs are differentiated in a directed differentiation protocol without the use of feeder cells. In the last step, cells dissociated from neurospheres are replated and cultured in the presence of neural differentiation medium. We found that it is preferred for the differentiated neurons to exhibit specific uptake of dopamine (and therefore be deemed as dopaminergic neurons) is to avoid formation of aggregates once the iPSC-derived neuroepithelial cells are plated in the neural differentiation medium. For example, we found that aggregates can be avoided by plating the cells on coating comprising extracellular matrix (ECM) material. While such extracellular matrix bed or layer can be prepared from cells, such material is also commercially available. A suitable example is MATRIGEl®. Another example is PATHCLEAR® (from AMS Biotechnology). Without intending to be bound by any particular theory, it is considered that the additional coating of ECM (such as MATRIGEL) inhibited the formation of globular cell aggregates, possibly by inhibiting cell migration. Preferably, the surface is coated with a combination of laminin, a polybasic amino acid and an extracellular matrix material. Polybasic amino acid coatings include polyornithine and polylysine. The resulting neuronal cultures are more adherent and unexpectedly exhibited specific dopamine uptake. Such cells were not produced when collagen was used for plating.

Neuronal differentiation medium for forcing pluripotent cells toward dopaminergic neurons generally comprises BDNF and GDNF. Thus, in one embodiment, the neuronal differentiation medium is neurobasal medium containing 1% N2 supplement, 1% MEM non-essential amino acids, 2% B27 supplement, 0.2 mM vitamin C, 1 μM cAMP, 1 μg/ml lamin, 1 ng/ml TGFβ3, 20 ng/ml Brain-derived neurotrophic factor (BDNF), 50 ng/ml glial cell line-derived neurotrophic factor (GDNF). Incubation for 14 days is generally required for the cells to begin exhibiting neuronal characteristics. We found that dopaminergic neurons could be obtained after 14 days in culture. The cells can be identified as being neurons by electrophysiological properties and/or by specific markers. For example, dopaminergic neurons can be identified by staining for the presence of tyrosine hydroxylase, or by specific uptake of dopamine. We observed that TH staining could be observed at or after 14 days in the neuronal differentiation medium, action potential at or after 1 month, DA uptake at or after 1 month.

Those skilled in the art will recognize, given the benefit of the present disclosure, that control and parkin neurons made as described herein can be used to screen for compounds that can enhance specific dopamine uptake. For example, populations of cells comprising neurons prepared by the process described herein could be used to analyze specific dopamine uptake through the dopamine transporter (DAT) which is critical for maintaining the temporal and spatial precision of dopaminergic transmission. In this regard, we have already demonstrated that parkin enhances dopamine uptake by degrading misfolded DAT. (H. Jiang, Q. Jiang and J. Feng (2004). Parkin Increases Dopamine Uptake by Enhancing the Cell Surface Expression of Dopamine Transporter. J. Biol. Chem. 279:54380-54386). Therefore, test agents that are determined to either enhance the folding of DAT or enhance the degradation of misfolded DAT in parkin neurons could improve DAT function to a level similar to that in control neurons made as described herein. Such compounds are considered to be candidates for use in PD therapy. Thus, in one embodiment, the invention provides a method for determining whether a test agent is a candidate for use in familial PD comprising contacting a cell population comprising parkin neurons with a test agent and determining an increase in specific dopamine uptake relative to parkin neurons that have not been contacted with the test agent. An increase in dopamine uptake is indicative that the test agent is a candidate for use in familial PD therapy.

The invention also provides an additional method for identifying agents as candidates for use in familial PD therapy. This embodiment entails analyzing test agents for the capability to affect monoamine oxidase (MAO) expression, such as a reduction in MAO mRNA. MAOs are mitochondrial enzymes responsible for the oxidative deamination of dopamine.

Any suitable technique for determining changes in MAO transcription in response to a test agent can be used in the method of the invention. In one embodiment, MAO-A and/or MAO-B mRNAs are measured by RT-PCR. In another embodiment, luciferase reporters under the control of MAO-A and/or MAO-B promoter can be inserted into the cells to measure the transcription of MAO-A and/or MAO-B genes. Test agents that can suppress the transcription of MAO-A and/or MAO-B in parkin neurons could restore the MAO levels to those in control neurons. Such compounds are considered to be candidates for use in PD therapy. Thus, in one embodiment, the invention provides a method for determining whether a test agent is a candidate for use in PD therapy by contacting parkin neurons with the test agent to see whether MAO-A and/or MAO-B transcription is suppressed to the level similar to that in control neurons.

In connection with MAO expression, we have now determined that parkin controls oxidative stress by limiting the expression of MAO by increasing the ubiquitination and degradation of Estrogen-Related Receptors (ERR), which are orphan nuclear receptors that play critical roles in the transcription regulation of many nuclear-encoded mitochondrial proteins. We have discovered that all three ERRs (α, β and γ) increase the transcription of MAO A and B and have determined that these effects are abolished by normal parkin protein, but not its PD-linked mutants. We have shown that parkin binds to ERRs and increases their ubiquitination and degradation. However, in fibroblasts from PD patients with parkin mutations or brain slices from parkin knockout mice, degradation of ERRs is significantly attenuated. This reveals the molecular mechanism by which parkin suppresses the transcription of monoamine oxidases to control oxidative stress induced by dopamine oxidation. Accordingly, in one embodiment of the invention, agents that can enhance ubiquitination and degradation of ERRs (α, β and γ and combinations thereof) are considered candidates for use in parkin PD therapy. The activity of these agents can be assessed by measuring changes in degradation of the ERRs, or by measuring changes in MAO transcription. Agents that can inhibit the transcription activation activity of ERRs are also considered candidate for use in PD therapy.

The following examples are provided to further illustrate the invention. These examples are not intended to be limited.

### EXAMPLE 1

### Vectors

Vectors comprising the reprogramming factors were constructed and inserted into cells as follows: FUW-tetO-LoxP-hOCT4, hSOX2, and hKLF4, FUW-M2rtTA, pMD2.G, psPAX2 and pSin-EF2-Nanog-Puromycin were purchased from Addgene of Cambridge, Mass., USA. Human c-Myc cDNA was purchased from THERMO SCIENTIFIC OpenBiosystems.

FUW-tetO-LoxP-hc-Myc: human c-Myc was amplified by PCR with human c-Myc cDNA as template. FUW-tetO-Loxp-hOCT4 was digested with EcoR I, and hOCT4 was removed and the vector was ligated with c-Myc PCR product to produce a new construct, FUW-tetO-Loxp-hc-Myc. The sequence was verified by sequencing.

FUW-tetO-Loxp-hNanog: human Nanog was digested from pSin-EF2-Nanog-Puromycin with Spe I/EcoR I. FUW-tetO-Loxp-hOCT4 was digested with EcoR I, and hOCT4 was removed and the vector was ligated with human Nanog fragment to produce a new construct, FUW-tetO-Loxp-hNanog. The sequence was verified by sequencing.

FUW-LoxP-M2rtTA: a fragment containing a loxP site was digested from FUW-tetO-Loxp-hOCT4 with BspEI and inserted into the BspEI site of the 3′LTR of FUW-M2rtTA. This construct results in a modified FUW-M2rtTA vector that has a LoxP site so that the m2rtTA can be removed from the genome of the iPSCs or the parkin neurons or the control neurons if necessary or desired.

### Production of Virus

For virus production in 293FT cells, the following steps were followed. For each 10-cm dish, 10 ug of FUW-tetO-LoxP-cDNA (hOCT4, hSOX2, hKLF4, c-Myc and Nanog) or 10 ug of FUW-LoxP-M2rtTA plasmid with 2.5 ug of pMD2.G and 7.5 ug pf psPAX2 were used. The plasmids were aliquoted into a tube and mixed well. 30 ul of Fugene transfection reagent was delivered into a second tube containing 500 ul of DMEM without serum and mixed gently and incubated for 5 min at RT. The two fractions were mixed and incubated for 20 min at RT. The DNA/Fugene 6 complex was added into the dish of 293FT cells. It was evenly distributed and incubated overnight at 37C. The transfection reagent-containing medium was aspirated, fresh collection medium was added, and the cells were returned to the incubator. The medium from the 293FT cells was collected 60 hours after transfection by using a 10-ml sterile pipet. The titer of the virus was measured using a commercially available ELISA kit for p24—the lentiviral capsid protein (ZEPTOMETRIX Corporation, Buffalo, N.Y.).

Human Fibroblasts: “P001” refers to fibroblasts from a parkin PD patient with compound heterozygous deletion of exon 3 and exon 5 of parkin. “P002” refers to a parkin PD patient with homozygous deletion of exon 3 of parkin. Fibroblasts from individuals having parkin PD that is associated with any other parkin mutations can also be used in the invention. “C001” refers to fibroblasts from a normal subject (unaffected with PD, unrelated spouse of an idiopathic PD patient). “C002” refers to a normal subject (unaffected PD, unrelated spouse of an idiopathic PD patient). We generated several lines of iPSCs for each fibroblast line and studied one line for each human subject (P001 clone #3; P002 clone #4.5; C001 clone #2; C002 clone #1).

Infection of Fibroblasts with Lentivirus

To infect human fibroblasts with lentivirus containing reprogramming factors the following steps were followed. The fibroblasts were seeded at 1×10⁵ cells per well in 6-well plate 24 hours before infection. Cells were infected overnight in the presence of 4 ug/ml polybrene with: FUW-Lox-hOCT4 (multiplicity of infection “MOI”=15); FUW-Lox-hSOX2 (MOI=15); FUW-Lox-hKLF4 (MOI=15); FUW-Lox-hNanog (MOI=15); FUW-Lox-h c-Myc (MOI=6); FUW-M2rtTA (MOI=30). Virus were removed next day, and washed with fresh medium. One day later the infected fibroblasts were split and plated onto 10-cm dish with feeders at 1×105 cells/dish in iPSC medium (DMEM/F12 with 20% knockout serum replacement, 0.1 mM 2-mercaptoethanol, 0.1 mM MEM nonessential amino acids, 1 mM L-glutamine and 4 ng/ml FGF2).

The cells were incubated with 1 ug/ml DOX for at least 9 days, and then the concentration was changed to 0.5 ug/ml DOX in iPSC medium on day 10. Incubation was continued at the same concentration until iPSCs appear. These cells have unique morphology easily recognized by those skilled in the art. For example, iPSCs grow larger than other cells in a population of cells that comprise iPSCs and non-iPSCs. In some cultures, 0.5-1 mM Valproic acid (VPA), a histone deacetylase inhibitor, was added as it was observed to increase iPS efficiency. On day 14-40 clones with iPSC morphology appeared. These clones are flat, highly refractory under a phase contrast microscope. They form tight, round colony and have a high nucleus/cytoplasm ratio. They also grow larger and large whereas other cells in the dish do not grow at that stage. Clones were picked for expansion and characterization.

Unlike the procedure described in Soldner et al. we did not remove the polynucleotides encoding the reprogramming factors or the M2rtTA sequence from the iPSCs.

The iPS cells produced as set forth above expressed the pluripotency markers alkaline phosphatase (AP), Oct4, Nanog, SSEA3, SSEA4, Tra-1-60 and Tra-1-81. RT-PCR analysis showed that viral transgenes were significantly silenced A panel of endogenous pluripotency genes were turned on to similar extents as those in H9 hES cells.

### EXAMPLE 2

This example describes the process to direct differentiation of the iPSCs into dopaminergic neurons. iPSC cultures were dissociated by treatment with Dispase (1 mg/ml in DMEM/F12 medium) or Collagenase IV (at the same concentration). Colonies of cells were dislodged from the cultures dishes and were recovered to form cell clusters in suspension culture. The cells were resuspended in iPSC medium containing a TGFβ inhibitor (10 uM SB431542, which is 4-[4-(1,3-benzodioxol-5-yl)-5-(2-pyridinyl)-1H-imidazol-2-yl]benzamide, a potent and selective inhibitor of TGF-β type I receptor activin receptor-like kinases). TGFβ inhibitors such as SB431542 will enhance the differentiation of iPSC to cells of neural lineage. After the iPS cell clusters are cultured in iPSC media for 4 days in free floating culture, the clusters are differentiated to neuroepithelia cells. The clusters are cultured for two days in suspension in Neural Induction Media (DMEM/F12 contains 1% N2 supplement, 0.1 mM NEM Non-essential amino acids solution, and 2 ug/ml Heparin) supplemented with 20 ng/ml bFGF. Clusters were plated on plates coated with laminin or extracellular matrix sold under the trade-name MATRIGEL. After 4-5 days following attachment of cell clusters, colonies will take on a morphology in which the center cells exhibit an elongated, columnar morphology (rosette). These rosettes will be cultured in Neural Induction Medium supplemented with FGF8 (20 ng/ml) and SHH (100 ng/ml) for one week. Then the rosettes were collected following dispase treatment. The rosettes peel off and form neurospheres in suspension culture. Neurospheres were cultured in suspension for 6 days in neural induction medium containing FGF8 (50 ng/ml), SHH (100 ng/ml), B27 (1×) and ascorbic acid (200 uM).

The neurospheres were dissociated with accutase and trypsin. The cells were evenly plated onto culture dishes or glass coverslips precoated first with polyornithine, then laminin, then matrigel. We determined it is important for the coverslip and plate to coated with 2% Matrigel (similar substances are also expected to be suitable) for 2 h at room temperature before plating cells).

The cells attached in 1-2 hr. Then 400 ul for each coverslip and 1.2 ml for each well of 6 well plates of neural differentiation medium containing FGF8 (50 ng/ml), Sonic Hedgehog Homolog (SHH), a growth factor important for brain development (100 ng/ml), B27 (1×), ascorbic acid (200 uM), cAMP (1 uM), laminin (1 ug/ml), TGFβ3 (1 ng/ml) and trophic factors (BDGF 20 ng/ml, GDNF 50 ng/ml) in DMEM was added.

Differentiation was continued by feeding the culture with the neuronal differentiation medium every other day (half medium change). 6 days later, FGF8 was withdrawn. SHH was decreased to 10 ng/ml after 14 days differentiation (about 38 days of total differentiation). At this point, invidual neuroepithelial cells often reaggregate to form small monolayer rosettes with differentiating neurons surrounding the clusters. In additional four weeks (66 days since start), more mature dopamine neurons appear.

### EXAMPLE 3

This example describes the characterization of dopaminergic neurons obtained in Example 2. Immunostaining of the cultures reveal numerous TH neurons expressing En-1, the transcription factor expressed by midbrain cells. Many of the TH neurons exhibit a complex neuronal morphology. The TH neurons also express AADC, VMAT2, DAT, which are markers for mature dopaminergic neurons. The neurons also express markers for mature neurons such as MAP2 and βIII-tubulin, as well as synaptic markers, which include but are not necessarily limited to NR1, synaptophysin, and PSD95, which show that they form synaptic connections with each other. Electrophysiological studies show that the neurons fire spontaneous action potentials, have spontaneous EPSC (Evoked Postsynaptic Currents), Na+ currents, K+, NMDA-evoked currents, and GABA-evoked currents. This shows that they behave like normal neurons in vivo and exhibit spontaneous and evoked synaptic activities. These neurons also release dopamine upon membrane depolarization (90 mM KCl treatment), and dopamine release is significantly blocked in calcium free buffer. These data show that the iPSC-derived dopaminergic neurons release dopamine in an activity-dependent manner.

It will be recognized from the foregoing that we obtained the novel iPSC-derived dopaminergic neurons in part by making important improvements to previously available iPSC engineering and culturing techniques to produce dopaminergic neurons with unexpected properties that closely mimic dopaminergic neurons in vivo. Our improvements include insertion of a polynucleotide encoding Nanog and an M2rtTA sequence flanked with LoxP sites to create iPSCs, non-removal of polynucleotides encoding the reprogramming factors and M2rtTA (although removal is possible due to LoxP sites), use of SB431542 at day 1-5 to increase the production of dopaminergic neurons, and the use of extracellular matrix bed (in addition to polyornithine and laminin) to minimize the formation of cell aggregates. Thus, the invention permits specific dopamine uptake to be measured for the first time in neurons derived from induced pluripotent stem cells or human embryonic stem cells.

### EXAMPLE 4

This example describes demonstration of specific dopamine uptake in the dopaminergic neurons obtained in Example 3.

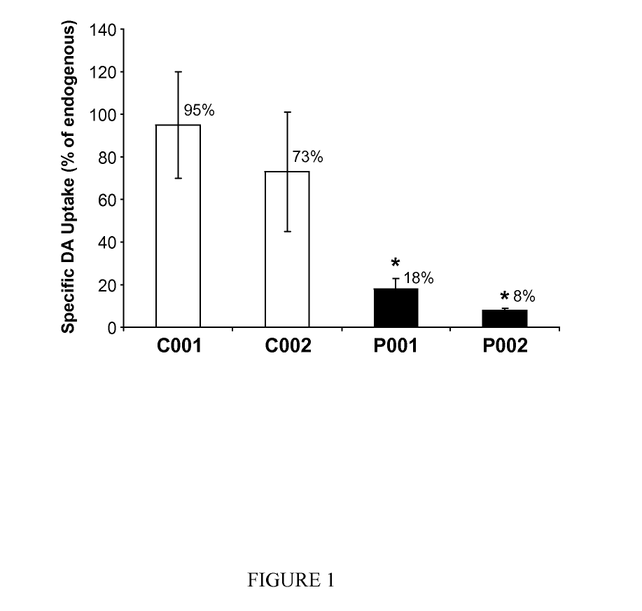

Dopamine uptake was measured by HPLC. Briefly, iPSC-derived neuronal cultures made as described above were rinsed with 1 ml of prewarmed uptake buffer (10 mM HEPES, 130 mM NaCl, 1.3 mM KCl, 2.2 mM CaCl2, 1.2 mM MgSO4, 1.2 mM KH2PO4, 10 mM glucose, pH 7.4) three times. Cells were incubated in 1 ml uptake buffer in the presence or absence of 10 uM Nomifensine (a specific inhibitor of dopamine transporter) for 10 min at 37° C. Then 5 μM dopamine was added and incubated for another 10 min. This represents three conditions done in parallel: (a) 1 ml uptake buffer alone; (b) 1 ml uptake buffer containing 5 μM dopamine; (c) 1 ml uptake buffer containing 5 μM dopamine and 10 μM nomifensine. After 10 minutes, cultures were washed at least three times in uptake buffer and lysed in 0.1 M perchloric acid with 1 mM EDTA, and 0.1 mM sodium bisulfite. Cleared cell lysates were analyzed for dopamine on HPLC coupled with electrochemical detection. Proteins in the pellets were dissolved in 1 ml 0.5N NaOH and protein content was measured using DC protein assay kit. The amount of dopamine detected by HPLC is first normalized against total cellular protein contents, which were very similar between different samples. Specific dopamine uptake is reflected in (c)-(b). Because the amount of endogenous dopamine in (a) reflects the number of dopaminergic neurons in the culture, normalized specific dopamine uptake is calculated using the formula [(c)-(b)]/(a), which is shown in FIG. 1. We found that iPSC-derived neurons from two control subjects showed robust specific dopamine uptake, while specific dopamine uptake in iPSC-derived neurons from two PD patients with parkin mutations (parkin neurons) was greatly diminished.

### EXAMPLE 5

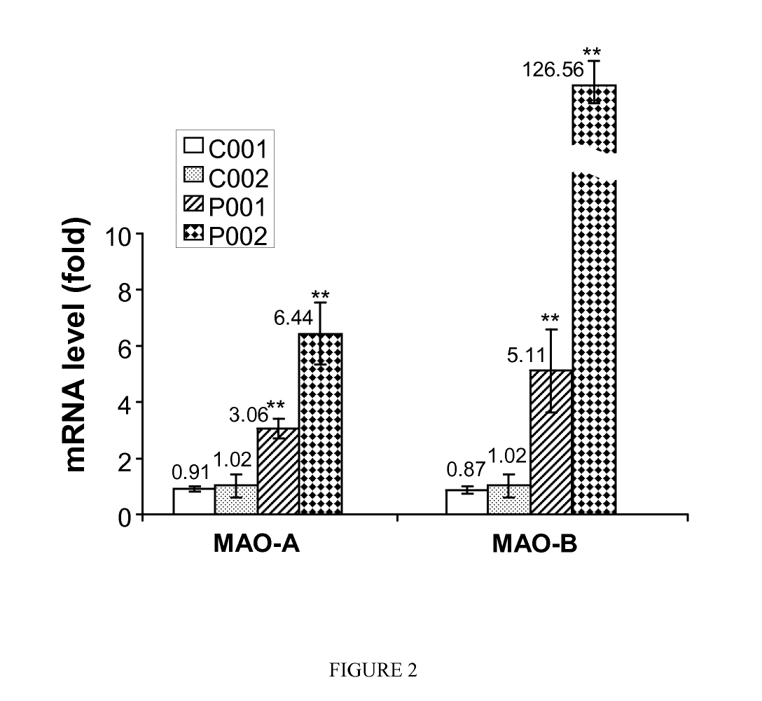

This examples demonstrates that mRNA levels for MAO can be quantified in iPSC derived dopaminergic neurons. For this experiment, dopaminergic neurons were differentiated from iPSCs derived from control subjects (C001 and C002) or PD patients with parkin mutations (P001 and P002). We measured the mRNA levels of monoamine oxidase A (MAO-A) and monoamine oxidase B (MAO-B) by quantitative real-time RT-PCR. The mRNA level of GAPDH was used as an internal control, which showed very similar expression levels in the four iPSC-derived neuronal cultures. As shown in FIG. 2, the amounts of MAO-A mRNA were significantly increased in iPSC-derived neurons from PD patients with parkin mutations (parkin neurons), compared to those from the two normal control subjects (control neurons). The amounts of MAO-B mRNA were also greatly increased. Thus, these dopaminergic neurons can be used to test potential candidates useful as therapeutic agents for disorders related to deficiency or malfunction of dopaminergic system.

While the present invention has been shown and described with reference to certain preferred embodiments thereof, it will be understood by those skilled in the art that various changes in form and details may be made therein without departing from the spirit and scope of the invention as described.

We claim:

1. A population of cells in culture comprising neuronal cells derived from induced pluripotent stem cells, said neuronal cells exhibiting specific uptake of dopamine.

2. The population of cells in culture from claim 1, wherein the induced pluripotent cells are derived from fibroblasts obtained from an individual who does not have familial Parkinson's disease.

3. The population of cells in culture from claim 2, wherein said neuronal cells exhibit specific uptake of dopamine that is at least 50% over endogenous levels of dopamine.

4. A method for obtaining induced pluripotent stem cells (iPSCs) comprising:
a) obtaining fibroblasts from an individual;
b)introducing Oct4, Sox2, Klf4, c-Myc and Nanog genes into the fibroblasts to obtain iPSCs.

5. The method of claim 4, wherein the fibroblasts are obtained from an individual who does not have familial Parkinson's disease and the iPSCs are capable of differentiating into dopaminergic neurons capable of specific dopamine uptake.

6. The method of claim 4, wherein the fibroblasts are obtained from an individual who has familial Parkinson's disease.

7. The method of claim 4, wherein the fibroblasts are skin fibroblasts.

8. A method for obtaining cells of neuronal lineage comprising:
a) obtaining iPSCs from the method of claim 4;
b) dissociating the iPSCs to obtain a cell suspension;
c) plating cells on a surface coated with materials such that the cells generate dopamine neurons capable of selective dopamine uptake.

9. The method of claim 8, wherein the cells are plated on a surface coated with laminin, a polybasic amino acid and extracellular matrix.

10. The method of claim 9, wherein the polybasic amino acid is polyornithine or polylysine.

11. The method of claim 9, wherein the iPSCs are derived from an individual who does not have idiopathic Parkinson's disease, and wherein the cells of neural lineage after step c) exhibit specific dopamine uptake.

12. A method for identifying a test agent as a potential candidate for reducing the severity of Parkinson's disease (PD) comprising:
a) obtaining cells of neural lineage according to the method of claim 9, from an individual who has PD;
b)incubating one sample of the cells with the test agent and one sample without the test agent;
c) measuring dopaminergic characteristics in both the samples, wherein the dopaminergic characteristics are selected from the group consisting of specific dopamine uptake, MAO transcription levels, protein and/or activity levels of estrogen-related receptors and combinations thereof;
wherein an increase in specific dopamine uptake, inhibition of MAO transcription or decrease in the level and/or activity of estrogen-related receptors is indicative of a potential candidate for reducing the severity of PD.
